(19) United States
(12) Patent Application Publication
(10) Pub. No.: US 2022/0062285 A1
(43) Pub. Date: Mar. 3, 2022

(54) METHOD OF TREATING A SKIN DISORDER WITH EGFR INHIBITOR

(71) Applicant: Sol-Gel Technologies Ltd., Ness Ziona (IL)

(72) Inventors: Moshe ARKIN, Kfar Shmaryahu (IL); Marcel ZIGHELBOIM, Kiryat Motzkin (IL); Ofra LEVY-HACHAM, Ness Ziona (IL); Ori NOV, Tarum (IL)

(73) Assignee: Sol-Gel Technologies Ltd., Ness Ziona (IL)

(21) Appl. No.: 17/521,884

(22) Filed: Nov. 9, 2021

Related U.S. Application Data
(63) Continuation-in-part of application No. PCT/IL2020/051153, filed on Nov. 5, 2020.
(60) Provisional application No. 62/931,252, filed on Nov. 6, 2019.

(51) Int. Cl. A61K 31/517; A61K 45/06; A61K 9/00; A61K 9/06

(52) U.S. Cl. CPC A61K 31/517; A61K 9/06; A61K 9/0014; A61K 45/06

(57) ABSTRACT

The present invention, in some embodiments, relates to a method of treatment, prevention or alleviation of Palmoplantar Keratoderma (PPK) or Olmsted syndrome in a patient in need thereof, comprising a debridement step followed by topical administration of a composition comprising at least one Epidermal Growth Factor Receptor (EGFR) inhibitor.

### FIELD OF THE INVENTION

The present invention, in some embodiments, relates to a method of treatment, prevention or alleviation of Palmoplantar Keratoderma (PPK) or Olmsted syndrome in a patient in need thereof, comprising a debridement step followed by topical administration of a composition comprising at least one Epidermal Growth Factor Receptor (EGFR) inhibitor.

### BACKGROUND OF THE INVENTION

### Epidermal Growth Factor Receptor (EGFR) Inhibitor Drugs

Epidermal Growth Factor Receptor (EGFR) inhibitor drugs like erlotinib, gefitinib, osimertinib and brigatinib target the EGFR and are used for the systemic treatment of some forms of cancer (lung, colon).

There is no US-marketed EGFR inhibitor drug for topical use. The EGFR inhibitor erlotinib is sold as oral tablets (Tarceva). Similarly, gefitinib (Iressa), osimertinib (Tigresso) and brigatinib (Alunbrig) are sold as oral tablets.

Treatment with EGFR inhibitors is known to induce cutaneous conditions like acneiform rash, papulopustular rash, abnormal scalp hair growth, abnormal facial hair growth, abnormal hair growth, abnormal eyelash growth, paronychia with or without pyogenic granulomas and telangiectasia.

### Palmoplantar Keratoderma (PPK)

Palmoplantar keratodermas (PPKs) represent a diverse group of hereditary and acquired disorders of keratinization in which there is hyperkeratosis of the palms and soles. Inherited PPKs are classified according to their morphology and distribution into four broad categories:
diffuse PPK characterized by uniform involvement of the palmoplantar surface;
focal PPK characterized by localized areas of hyperkeratosis mainly over pressure points;
striate PPK consisting of linear lesions mostly appreciable at the volar aspect of the fingers and palms in correspondence of the underlying tendons; and
punctate PPK is characterized by small (1-10 mm) keratotic papules on the palmoplantar surface.

### Punctate Palmoplantar Keratoderma Type 1

Punctate palmoplantar keratoderma type 1 (PPPK-1) is a rare autosomal dominant inherited skin disease characterized by multiple hyperkeratotic papules involving the palms and soles and may be seen in the skin creases, mostly in those of African descent. Signs and symptoms of punctate palmoplantar keratoderma type 1 tend to become evident between the ages of 10 to 30 years. Symptoms include multiple tiny, hard, round bumps of thickened skin on the palms of the hands and soles of the feet. These bumps may join to form calluses on pressure points, which may cause pain. They may also make walking difficult or impair hand or finger movement. Symptoms tend to worsen with time and may be aggravated by manual work or injury. The lesions begin as pinpoint firm papules which may be translucent or become opaque or verrucous over time. This gives PPPK-1 a clinical appearance distinct from focal or diffuse PPK. In mechanically irritated areas, confluent callus-like plaques can be found and may be painful. Unlike in other keratodermas, lesions are uncommon in childhood and usually develop in adolescence and adulthood.

Pain experienced by subjects having PPK might be at least temporarily decreased following a physical debridement of the area affected by the PPK, however the pain usually comes back after a quite short period of time (above 6 weeks) (Redmond et al. J. Am. Podiatr. Med. Assoc. 89(10): 515-519, 1999).

### Olmsted Syndrome

Olmsted syndrome (OS) is a rare genodermatosis classically characterized by the combination of bilateral mutilating transgredient palmoplantar keratoderma (PPK) and periorificial keratotic plaques, but which shows considerable clinical heterogeneity. The disease starts usually at birth or in early childhood.

There is an unmet need for efficient and patient-friendly methods of treatment, prevention or alleviation of palmoplantar keratoderma and for Olmsted syndrome.

### SUMMARY OF THE INVENTION

In one aspect this invention provides a method of treatment, prevention or alleviation of Palmoplantar Keratoderma (PPK) or Olmsted syndrome in a patient in need thereof, comprising:
a. debridement of affected PPK surface area or Olmsted Surface area of said patient; and
b. topical administration, following the debridement of step (a), of a therapeutically effective amount of a topical composition comprising at least one Epidermal Growth Factor Receptor (EGFR) inhibitor, to said affected surface areas.

### DETAILED DESCRIPTION OF THE INVENTION

In one aspect, this invention provides a method of treatment, prevention or alleviation of Palmoplantar Keratoderma (PPK) or Olmsted syndrome in a patient in need thereof, comprising:
a. debridement of affected PPK surface area or Olmsted syndrome surface area of said patient; and
b. topical administration, of a therapeutically effective amount of a topical composition comprising at least one Epidermal Growth Factor Receptor (EGFR) inhibitor, to said affected surface areas;
   wherein the debridement step and the topical administration can be sequentially, concomitantly, or the topical administration is only applied after the debridement process is completed.

In one aspect, this invention provides a method of treatment, prevention or alleviation of Palmoplantar Keratoderma (PPK) or Olmsted syndrome in a patient in need thereof, comprising:
a. debridement of affected PPK surface area or Olmsted syndrome surface area of said patient; and
b. topical administration, following the debridement of step (a), of a therapeutically effective amount of a topical composition comprising at least one Epidermal Growth Factor Receptor (EGFR) inhibitor, to said affected surface areas.

In some embodiments, the topical composition comprising at least one Epidermal Growth Factor Receptor (EGFR) inhibitor is administered for at least one week. In some embodiments, the topical composition is administered for 12 to 14 weeks, preferably administered for 12 weeks. In some embodiments, the topical composition comprising at least one Epidermal Growth Factor Receptor (EGFR) inhibitor is administered once daily for 12 weeks. Each possibility represents a separate embodiment of this invention.

In one embodiment, the treatment, prevention or alleviation of PPK or Olmsted syndrome by topical administration of a composition comprising at least one EGFR inhibitor does not induce or induces reduced cutaneous side-effects as compared with systemic administration of the same EGFR inhibitor amount. Non-limiting examples of side effects of erlotinib (oral tablet) include infection, conjunctivitis, diarrhea, dyspnea, keratoconjunctivitis, nausea, pruritus, skin rash, stomatitis, anorexia, and xeroderma.

In one embodiment, the term “debridement” refers to removal of nonviable tissue such as dead, damaged, infected, or excessive tissue. The debriding process (“debridement”) is performed by physical, chemical/pharmacological, or mechanical removal of the nonviable or unwanted tissue. In some embodiments, the nonviable or unwanted tissue results from and/or is a symptom of the PPK, i.e. they are affected by the PPK. In some embodiments, the nonviable or unwanted tissue results from and/or is a symptom of the Olmsted syndrome. In some embodiments, the debridement is performed by physical, chemical/pharmacological or mechanical removal of nonviable or unwanted tissue affected by the PPK. In some embodiments, the debridement is performed by physical, chemical/pharmacological or mechanical removal of nonviable or unwanted tissue affected by the Olmsted syndrome.

In one embodiment, the debridement step of the methods of this invention is performed via any procedure known in the art. In another embodiment, the physical or mechanical removal comprises using scalpel, monofilament pads, forceps, scissors or any other medical apparatus, device or tool as known in the art. In another embodiment, the debridement step comprises skin graft techniques (e.g. split-thickness sole skin graft: Wang et al.; Ann. Plast. Surg. 2018 February; 80 (2S Suppl 1):S55-S58, the content of which is incorporated herein by reference). In another embodiment, the chemical/pharmacological debridement comprises oral treatment of at least one EGFR inhibitor or by applying a chemical agent to the PPK or Olmsted syndrome surface area. In another embodiment, the physical, chemical/pharmacological or mechanical removal and the skin graft techniques are combined with or performed following by application of a chemical agent to the affected PPK surface area or Olmsted syndrome surface area. Each possibility represents a separate embodiment of this invention. In some embodiments, the skin graft techniques are combined with or performed following application of a chemical agent to said affected PPK surface area. In some embodiments, the skin graft techniques are combined with or performed following application of a chemical agent to said affected Olmsted syndrome surface area.

In some embodiments, the chemical/pharmacological debridement for removal of nonviable or unwanted tissue affected by the PPK comprises an oral administration of at least one EGFR inhibitor or by administering a chemical agent to the PPK surface area, or by combination thereof.

In some embodiments, the chemical/pharmacological debridement for removal of nonviable or unwanted tissue affected by the Olmsted syndrome comprises an oral administration of at least one EGFR inhibitor or administering a chemical agent to the Olmsted syndrome surface area, or by combination thereof.

The chemical or pharmacological debridement refers to inter alia administering an oral composition with at least one EGFR inhibitor, wherein the oral composition is administered sequentially, concomitantly or prior to the topical administration. In other embodiment the chemical or pharmacological debridement comprises administering an oral composition until the unwanted tissue disappears/removed, and only then a administering a topical composition. In another embodiment, the oral composition comprises erlotinib. In another embodiment, the topical composition comprises erlotinib. In another embodiment, both oral and topical compositions comprise erlotinib. In some embodiments, the chemical or pharmacological debridement comprises administering orally at least one EGFR inhibitor. Non limiting examples of EGFR inhibitor for oral administration include erlotinib hydrochloride. In other embodiment, erlotinib hydrochloride tablets, wherein the dosage of the erlotinib is between 25 mg to 150 mg. The dosage depends on patient's tolerability.

In another embodiment, the physical, chemical//pharmacological or mechanical removal is performed following applying a chemical agent to the affected PPK surface area.

In another embodiment, the physical, chemical//pharmacological or mechanical removal is performed following applying a chemical agent to the affected Olmsted syndrome surface area.

In another embodiment, the physical, chemical//pharmacological or mechanical removal is performed following, sequential, concomitant or prior to applying a chemical agent to the affected PPK surface area.

In another embodiment, the physical, chemical//pharmacological or mechanical removal is performed following, sequential, concomitant or prior to applying a chemical agent to the affected Olmsted syndrome surface area.

In another embodiment, “applying a chemical agent” as used herein is defined as soaking the dead, damaged, infected or excessive tissue in a chemical agent (e.g. a composition comprising the chemical agent), or injecting of the chemical agent to the tissue, or spreading the chemical agent to the affected PPK surface area or the Olmsted syndrome surface area or any other application method as known in the art. Each possibility represents a separate embodiment of this invention.

In another embodiment, the chemical agent used in the methods of this invention comprises urea (Mulay et al. AMA Arch Derm. 1958; 78(6):758, the content of which is incorporated herein by reference), salicylic acid, lactic acid, retinoids, psoralen, corticosteroids (Patel et al.; Am J Clin Dermatol 2007; 8 (1): 1-11, the content of which is incorporated herein by reference), debriding agent, derivatives thereof or any combination thereof.

In another embodiment, non-limiting examples of corticosteroids include: cortisol, corticosterone, cortisone, tixocortol, prednisolone, methyl prednisolone, budesonide, desonide, triamcinolone, betamethasone, mometasone, prednicarbate and aldosterone and any combination thereof.

In another embodiment, non-limiting examples of a retinoid include retinol, tretinoin, isotretinoin, alitretinoin, adapalene, bexarotene, tazarotene, etretinate, acitretin and any combination thereof.

In another embodiment, the chemical agent comprises a combination of urea, salicylic acid, lactic acid, retinoids, psoralen, corticosteroids or any combination thereof with a debriding agent. In another embodiment, psoralen is used in conjunction with UVA treatment. In another embodiment, non-limiting examples of the debriding agent include: debridase, vegetable preparation, bromelain, ananain, cysteine protease precursor and any combination thereof (U.S. Pat. Nos. 7,128,719, 8,119,124 and 8,540,983 and US Publication 2019/030140, the contents of which are incorporated herein by reference). Each possibility represents a separate embodiment of this invention.

In some embodiments, the debridement step comprises ultrasonication combined with application of a chemical agent to the affected PPK surface area. In some embodiments, the debridement step comprises ultrasonication combined with application of a chemical agent to the affected Olmsted syndrome surface area. In another embodiment, the debridement step comprises ultrasonication combined with application of a chemical agent e.g. a debriding agent (U.S. Pat. No. 7,128,719, the content of which is incorporated herein by reference). In another embodiment, debridement step comprises applying of a chemical agent, e.g. a debriding agent such as ananain, cysteine protease precursor, bromelain or any combination thereof (U.S. Pat. Nos. 8,119,124 and 8,540,983 and US Publication 2019/030140, the contents of which are incorporated herein by reference). In another embodiment, debridement step comprises applying a hydrogel composition comprising bromelain, ananain and a water-soluble gelling agent (US Publication 2019/142910). In another embodiment, the debridement step (as chemical/pharmaceutical debridement) comprises oral administration of at least one EGFR inhibitor. In another embodiment, the debridement step comprises oral administration of at least one EGFR inhibitor and optionally with retinoids. In some embodiments, the at least one EGFR inhibitor is erlotinib hydrochloride. In another embodiment, the topical composition used in step b) of the methods of this invention is a combination product comprising the EGFR and a chemical agent; or the EGFR and the chemical agent are administered sequentially or concomitantly (as separate products), where the EGFR and the chemical agent are as described hereinabove. In another embodiment, the physical, chemical/pharmacological mechanical removal or the skin graft techniques are performed prior or following administering the combination product, the EGFR or the chemical agent. Each possibility represents a separate embodiment of this invention.

In one embodiment, the topical composition used within the methods of this invention further comprises a therapeutically effective amount of at least one penetration enhancer. In another embodiment, the at least one penetration enhancer is selected from the group consisting of: dimethyl sulfoxide (DMSO), ethanol, isopropyl alcohol, dimethyl isosorbide, isopropyl myristate, oleic acid, a polyethylene glycol, hexylene glycol, glycofurol and combinations thereof.

In another embodiment, the amount of the at least one penetration enhancer is from about 10% to about 98% w/w of at least one penetration enhancer. In another embodiment, the amount of the at least one penetration enhancer is from about 10% to about 90% w/w of at least one penetration enhancer. In another embodiment, the amount of the at least one penetration enhancer is from about 10% to about 80% w/w of at least one penetration enhancer. In another embodiment, the amount of the at least one penetration enhancer is from about 10% to about 50% w/w of at least one penetration enhancer. In another embodiment, the amount of the at least one penetration enhancer is from about 10% to about 30% w/w of at least one penetration enhancer. In another embodiment, the amount of the at least one penetration enhancer is from about 30% to about 98% w/w of at least one penetration enhancer. In another embodiment, the amount of the at least one penetration enhancer is from about 50% to about 98% w/w of at least one penetration enhancer. In another embodiment, the amount of the at least one penetration enhancer is from about 70% to about 98% w/w of at least one penetration enhancer. In another embodiment, the at least one penetration enhancer has dual functionality and may act also as solvent. Each possibility represents a separate embodiment of this invention.

In one embodiment, the at least one EGFR inhibitor for oral treatment as chemical or pharmaceutical debridement as described hereinabove is selected from the group consisting of: erlotinib, gefitinib, lapatinib, osimertinib and brigatinib their salts, hydrates or solvates and combinations thereof. In another embodiment, the at least one EGFR inhibitor is erlotinib hydrochloride.

In one embodiment, the at least one EGFR inhibitor within the topical composition as described hereinabove is selected from the group consisting of: erlotinib, gefitinib, lapatinib, osimertinib and brigatinib their salts, hydrates or solvates and combinations thereof. In another embodiment, the at least one EGFR inhibitor is erlotinib hydrochloride. In another embodiment, the amount of at least one EGFR inhibitor is from about 0.1% w/w to about 10% w/w. In another embodiment, the amount of at least one EGFR inhibitor is from about 0.1% w/w to about 1% w/w. In another embodiment, the amount of at least one EGFR inhibitor is from about 0.1% w/w to about 2% w/w. In another embodiment, the amount of at least one EGFR inhibitor is from about 1% w/w to about 3% w/w. In another embodiment, the amount of at least one EGFR inhibitor is from about 3% w/w to about 5% w/w. In another embodiment, the amount of at least one EGFR inhibitor is from about 3% w/w to about 7% w/w. In another embodiment, the amount of at least one EGFR inhibitor is from about 5% w/w to about 10% w/w. In another embodiment, the at least one EGFR inhibitor is erlotinib hydrochloride and the amount thereof is 0.5% w/w. In another embodiment, the at least one EGFR inhibitor is erlotinib hydrochloride and the amount thereof is 0.75% w/w. In another embodiment, the at least one EGFR inhibitor is erlotinib hydrochloride and the amount thereof is 1% w/w. In another embodiment, the at least one EGFR inhibitor is erlotinib hydrochloride and the amount thereof is 1.25% w/w. Each possibility represents a separate embodiment of this invention.

A skilled artisan would appreciate that “a pharmaceutical acceptable salt” of EGFR inhibitor, may in some embodiments be formed by the reaction of an EGFR inhibitor compound with an acid or base. The term “pharmaceutically acceptable salt” may encompass those salts that retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxylic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. Other salts are known to those of skill in the art and can readily be adapted for use in accordance with the present compounds provided herein.

Suitable pharmaceutically-acceptable salts of amines of compounds provided herein may be prepared from an inorganic acid or from an organic acid. In some embodiments, examples of inorganic salts of amines are bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphate, sulfates, sulfamates, sulfanilates, sulfonic acids (alkylsulfonates, arylsulfonates, halogen substituted alkylsulfonates, halogen substituted arylsulfonates), sulfonates and thiocyanates.

In some embodiments, examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates gluconates, glutamates, glycolates, glucorate, glucoheptanoates, glycerophosphates, gluceptates, glycollylarsanilates, glutarates, glutamate, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, naphthalenesulfonates, 2-naphthalenesulfonates, nicotinates, nitrates, napsylates, N-methylglucamines, oxalates, octanoates, oleates, pamoates, phenylacetates, picrates, phenylbenzoates, pivalates, propionates, phthalates, phenylacetate, pectinates, phenylpropionates, palmitates, pantothenates, polygalacturates, pyruvates, quinates, salicylates, succinates, stearates, sulfanilate, subacetates, tartrates, theophyllineacetates, p-toluenesulfonates (tosylates), trifluoroacetates, terephthalates, tannates, teoclates, trihaloacetates, triethiodide, tricarboxylates, undecanoates and valerates.

In various embodiments, examples of inorganic salts of phosphite may be selected from ammonium, alkali metals to include lithium, sodium, potassium, cesium; alkaline earth metals to include calcium, magnesium, aluminium; zinc, barium, cholines, quaternary ammoniums.

In some embodiments, the salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

In one embodiment, the topical composition as described hereinabove further comprises at least one solvent. In another embodiment, the at least one solvent is selected from the group consisting of: DMSO, ethanol, isopropyl alcohol, propylene glycol, dimethyl isosorbide, isopropyl myristate, oleic acid, a polyethylene glycol, hexylene glycol, glycerin, glycofurol and combinations thereof. Each possibility represents a separate embodiment of this invention.

In some embodiments, the topical composition as described hereinabove further comprises at least one ingredient selected from the group consisting of: a moisturizer, a skin barrier, urea, ammonium lactate and combinations thereof, in a concentration of from about 0.01% w/w to about 1% w/w, from about 1% w/w to about 3% w/w or from about 3% w/w to about 5% w/w. Each possibility represents a separate embodiment of this invention.

In another embodiment, the method of treatment, prevention or alleviation of Palmoplantar Keratoderma (PPK) as described herein comprises a topical administration step of a topical composition in a patient in need thereof, comprising at least one Epidermal Growth Factor Receptor (EGFR) inhibitor, to an affected surface areas.

In another embodiment, the method of treatment, prevention or alleviation of Olmsted syndrome as described herein comprises a topical administration step of a topical composition in a patient in need thereof, comprising at least one Epidermal Growth Factor Receptor (EGFR) inhibitor, to an affected surface areas.

In some embodiments, the topical composition as described hereinabove further comprises at least one additional active agent from group 1, wherein the at least one additional active agent is selected from: tapinarof, a Janus kinase inhibitor (JAK inhibitor), a phosphodiesterase-4 inhibitor (PDE4 inhibitor), a corticosteroid, calcipotriene and combinations thereof, in a concentration of from about 0.01% w/w to about 1% w/w, from about 1% w/w to about 3% w/w or from about 3% w/w to about 5% w/w.

In another embodiment, non-limiting examples of JAK inhibitor include: ruxolitinib, oclacitinib, peficitinib, upadacitinib, fligotinib, momelotinib, pacritinib, tofacitinib, cucurbitacin-I and any combination thereof.

In another embodiment, non-limiting examples of PDE4 inhibitor include: apremilast, crisaborole, diazepam, luteolin, piclamilast, roflumilast and any combination thereof. In another embodiment, at least one EGFR inhibitor and said at least one additional active agent exhibit an additive or synergistic effect. Each possibility represents a separate embodiment of this invention.

In some embodiments, the topical composition as described hereinabove further comprises at least one additional active agent from group 2, wherein the at least one additional active agent is selected from menadione, ketoconazole, dapsone, cevimeline, spironolactone, retinoid, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% w/w to about 1% w/w, from about 1% w/w to about 3% w/w, from about 3% w/w to about 5% w/w or from about 5% w/w to about 10% w/w. In one embodiment, at least one EGFR inhibitor and said at least one additional active agent exhibit an additive or synergistic effect. Each possibility represents a separate embodiment of this invention.

In some embodiments, the topical composition comprises at least one Epidermal Growth Factor Receptor (EGFR) inhibitor, in combination with at least one active agent of group 1. In some embodiments, the topical composition comprises at least one Epidermal Growth Factor Receptor (EGFR) inhibitor, in combination with at least one active agent of group 2.

In some embodiments, the topical composition comprises at least one Epidermal Growth Factor Receptor (EGFR) inhibitor, in combination with at least one active agent of group 1 and of group 2.

In another embodiment, the method of treatment, prevention or alleviation of Palmoplantar Keratoderma (PPK) or Olmsted syndrome as described herein comprises a topical administration step of a topical composition in a patient in need thereof, comprising at least one Epidermal Growth Factor Receptor (EGFR) inhibitor, to an affected surface areas. In some embodiments, the topical administration comprises administering at least one Epidermal Growth Factor Receptor (EGFR) inhibitor, at least one active agent of group 1, at least one active agent of group 2, or any combination thereof, wherein each of at least one Epidermal Growth Factor Receptor (EGFR) inhibitor, at least one active agent of group 1, at least one active agent of group 2 is administered as a separate composition. In another embodiment, the at least one Epidermal Growth Factor Receptor (EGFR) inhibitor and the at least one active agent of group 1 are formulated as a combination composition. In another embodiment, the at least one Epidermal Growth Factor Receptor (EGFR) inhibitor and the at least one active agent of group 2 are formulated as a combination composition.

In some embodiments, the topical composition used in the methods of this invention is a gel, a cream, an ointment, a hydrogel, an emulsion, an elixir, a suspension, a tincture, a paste, an aerosol, a sebum control product, a lotion, a spray, a shampoo, a patch, a foam or any other formulation suitable for topical administration. In another embodiment, the topical composition is a gel, a lotion, a cream or a foam. In another embodiment, Sebum control products may include ingredients selected from azelaic acid, salicylic acid, sulfur, nicotinamide, L-carnitine and combinations thereof. Each possibility represents a separate embodiment of this invention.

In some embodiments, the at least one EGFR inhibitor within the topical composition as described hereinabove is partly or entirely solubilized.

In some embodiments, the at least one EGFR inhibitor within the topical composition as described hereinabove is erlotinib hydrochloride and the composition is formulated as a topical gel.

In one embodiment, the composition as described hereinabove comprises about 0.75% w/w erlotinib hydrochloride and from about 10% to about 98% w/w of at least one penetration enhancer, and wherein the composition is formulated as a gel. In another embodiment, the composition comprises about 0.75% w/w erlotinib hydrochloride, about 70% w/w DMSO, about 25% propylene glycol, about 0.5% w/w 2-phenoxyethanol, about 0.25% w/w methylparaben and about 3% w/w Carbopol 980, wherein the composition is formulated as a gel.

In one embodiment, the composition as described hereinabove comprises from about 0.1% w/w to about 1% w/w, from about 1% w/w to about 3%, from about 3% w/w to about 5% w/w or from about 5% w/w to about 10% w/w erlotinib hydrochloride, from about 0.01% w/w to about 1% w/w, from about 1% w/w to about 3% w/w or from about 3% w/w to about 5% w/w tapinarof and from about 10% to about 98% w/w at least one penetration enhancer. Each possibility represents a separate embodiment of this invention.

In one embodiment, the composition as described hereinabove comprises from about 0.1% w/w to about 1% w/w, from about 1% w/w to about 3% w/w, from about 3% w/w to about 5% w/w or from about 5% w/w to about 10% w/w erlotinib hydrochloride, from about 0.01% w/w to about 1% w/w, from about 1% w/w to about 3% w/w or from about 3% w/w to about 5% w/w tofacitinib citrate and from about 10% w/w to about 98% w/w at least one penetration enhancer. Each possibility represents a separate embodiment of this invention.

In one embodiment, the composition as described hereinabove comprises from about 0.1% w/w to about 1% w/w, from about 1% w/w to about 3% w/w, from about 3% w/w to about 5% w/w or from about 5% w/w to about 10% w/w erlotinib hydrochloride, from about 0.01% w/w to about 1% w/w, from about 1% w/w to about 3% w/w or from about 3% w/w to about 5% w/w apremilast and from about 10% w/w to about 98% w/w at least one penetration enhancer. Each possibility represents a separate embodiment of this invention.

In one embodiment, the composition as described hereinabove is a topical gel composition comprising 0.75% Erlotinib HCl and 70% DMSO.

In one embodiment, the composition as described hereinabove is a topical gel composition comprising 0.5% Erlotinib HCl and 70% DMSO.

In one embodiment, the composition as described hereinabove is a topical gel composition comprising 0.5% Erlotinib HCl and 45.5% DMSO.

In one embodiment, the composition as described hereinabove is a topical gel composition comprising 0.5% Erlotinib HCl and 50% EtOH 70%.

In one embodiment, the composition as described hereinabove is a topical gel composition comprising 1.25% Erlotinib HCl and 95% DMSO.

In one embodiment, the composition as described hereinabove is a topical gel composition comprising 1% Erlotinib HCl, 49% PEG-400 and 30% PEG-3350.

In one embodiment, the composition as described hereinabove is a topical gel composition comprising 1% Erlotinib HCl and 1% Tapinarof.

In one embodiment, the composition as described hereinabove is a topical gel composition comprising 0.75% Erlotinib HCl and 0.5% Tofacitinib Citrate.

In one embodiment, the composition as described hereinabove is a topical gel composition comprising 0.75% Erlotinib HCl and 0.5% Apremilast.

In some embodiments, the PPK is acquired or hereditary. In some embodiments, the PPK is diffuse, focal, striate or punctate PPK. In another embodiment, the punctate PPK is a Punctate palmoplantar keratoderma type 1 (PPPK-1). In some embodiments the PPK comprises Epidermolytic palmoplantar keratoderma (EPPK), punctate PPK (non-limiting examples include: Type I, Type II, Type III), diffuse PPK (non-limiting examples include: Vörner PPK, Nagashima PPK, Bothnian PPK, Greither PPK, Sybery syndrome, Gamborg Nielsen PPK, Acral keratoderma, Huriez syndrome), Diffuse mutilating PPK, focal PPK (non-limiting example includes: PPK nummularis), striate PPK (non-limiting examples include: Striate PPK I, Striate PPK II, Striate PPK III), Each possibility represents a separate embodiment of this invention. In some embodiments, PPK is observed in patients having at least one of the following: keratins, desmosomes, gap junctions, connexins, loricrins or any combination thereof. In some embodiments, PPK with deafness is observed in patients having mutations in the GJB2 or MT-TS1 genes. In some embodiments, PPK with deafness is caused by mutations in the GJB2 or MT-TS1 genes. In some embodiments, Epidermolytic palmoplantar keratoderma (EPPK) is caused by Keratin 9 mutation. In some embodiments, PPK and deafness is observed in pedigrees patients with Mitochondrial A7445G mutation. In some embodiments, PPK and deaf-mutism is observed in patients with functional defects of Cx26 result from a heterozygous missense mutation. In some embodiments, Type 1 PPK striata (PPKS) is caused by heterozygous mutation in the DSG1 gene (125670) on chromosome 18q12. In some embodiments, Type II PPKS (PPKS2; 612908) is caused by mutation in the DSP gene (125647) on chromosome 6. In some embodiments Type III PPKS (PPKS3; 607654) is caused by mutation in the keratin-1 gene (KRT1; 139350) on chromosome 12q. In some embodiments, Nagashima type of palmoplantar keratoderma (PPKN) is caused by homozygous or compound heterozygous mutation in the SERPINB7 gene (603357) on chromosome 18q21.

In some embodiments, the methods as described hereinabove are used in the treatment of the following indications: calluses, corns, psoriasis, warts, nail disorders and diseases such as onychomycosis, onychogryphosis, choloronychia, nail dystrophy or any combination thereof. Each possibility represents a separate embodiment of this invention.

In one embodiment, the methods of this invention have no drug related serious adverse events or transient serious adverse events, in respect to the topical EGFR inhibitor administration.

In one embodiment, the methods of this invention result in minimal change in thickness of the plantar skin at the end of treatment compared to vehicle (control) treatment in view of baseline, as shown by the Confidence Interval (CI). In another embodiment, the confidence interval is at least 75%. In another embodiment, the confidence interval is at least 80%. In another embodiment, the confidence interval is at least 85%. In another embodiment, the confidence interval is at least 90%. In another embodiment, the confidence interval is at least 95%. In another embodiment, the confidence interval is at least 99%. The confidence intervals are calculated using the “exact” confidence intervals, computed by the method of Clopper and Pearson (Biometrika 26:404-413, 1934), which is based on a relationship between the F distribution and the binomial distribution.

In one embodiment, the methods of this invention result in at least 75% Confidence Interval (CI) in the proportion of subjects who report at least “minimally improved” as measured by the PRO (Patient Reported Outcome) questionnaire. In another embodiment, the confidence interval is at least 80%. In another embodiment, the confidence interval is at least 85%. In another embodiment, the confidence interval is at least 90%. In another embodiment, the confidence interval is at least 95%. In another embodiment, the confidence interval is at least 99%. The confidence intervals are calculated using the “exact” confidence intervals, computed by the method of Clopper and Pearson (Biometrika 26:404-413, 1934), which is based on a relationship between the F distribution and the binomial distribution.

In one embodiment, the methods of this invention result in at least 75% Confidence Interval (CI) in the numeric difference within subject in the thickness of the plantar skin at 8 weeks time point vs the vehicle (control) group. In another embodiment, the confidence interval is at least 80%. In another embodiment, the confidence interval is at least 85%. In another embodiment, the confidence interval is at least 90%. In another embodiment, the confidence interval is at least 95%. In another embodiment, the confidence interval is at least 99%. The confidence intervals are calculated using the “exact” confidence intervals, computed by the method of Clopper and Pearson (Biometrika 26:404-413, 1934), which is based on a relationship between the F distribution and the binomial distribution.

In one embodiment, the methods of this invention show that there is no significant difference within subject in the thickness of the plantar skin at 12 weeks vs 24 weeks time point of the active group as shown by the Confidence Interval (CI). In another embodiment, the confidence interval is at least 75%. In another embodiment, the confidence interval is at least 80%. In another embodiment, the confidence interval is at least 85%. In another embodiment, the confidence interval is at least 90%. In another embodiment, the confidence interval is at least 95%. In another embodiment, the confidence interval is at least 99%. The confidence intervals are calculated using the “exact” confidence intervals, computed by the method of Clopper and Pearson (Biometrika 26:404-413, 1934), which is based on a relationship between the F distribution and the binomial distribution.

In one embodiment, the methods of this invention result in at least 75% Confidence Interval (CI) in the numeric difference within subject in the thickness of the plantar skin at 12 weeks and at 24 weeks time points of active group compared to the vehicle (control) group. In another embodiment, the confidence interval is at least 80%. In another embodiment, the confidence interval is at least 85%. In another embodiment, the confidence interval is at least 90%. In another embodiment, the confidence interval is at least 95%. In another embodiment, the confidence interval is at least 99%. The confidence intervals are calculated using the “exact” confidence intervals, computed by the method of Clopper and Pearson (Biometrika 26:404-413, 1934), which is based on a relationship between the F distribution and the binomial distribution.

In some embodiments, the frequency of administration of the topical composition within the methods of this invention can be determined empirically. In one embodiment, non-limiting examples of administration include: once daily, twice daily, weekly, bi-weekly and monthly. In another embodiment, the administration is once daily or twice daily. In another embodiment, the administration is once daily. Each possibility represents a separate embodiment of this invention.

In some embodiments, the dosage frequencies of the topical composition within the methods of this invention can be gradually decreased over time and maintained at a steady dose suitable for long-term—six months, 1 year, 5 years, 10 years or more, up to lifelong administration to control the symptoms of PPK. In one embodiment, dosage administration can begin at from twice a day, to once a day, to two times a week, to once a week, to once every two weeks or less frequent than once every two weeks. Each possibility represents a separate embodiment of this invention.

In some embodiments, the dosage frequencies of the topical composition within the methods of this invention can be gradually decreased over time and maintained at a steady dose suitable for long-term—six months, 1 year, 5 years, 10 years or more, up to lifelong administration to control the symptoms of Olmsted syndrome. In one embodiment, dosage administration can begin at from twice a day, to once a day, to two times a week, to once a week, to once every two weeks or less frequent than once every two weeks. Each possibility represents a separate embodiment of this invention.

In some embodiment, a pharmaceutically and/or dermatologically acceptable vehicle is found within the compositions as described hereinabove. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream and hydrophilic ointment. In another embodiment, suitable pharmaceutically and dermatologically acceptable vehicles for topical application include lotions, creams, foams, solutions, gels, patches and the like. Generally, the vehicle is either organic in nature or an aqueous emulsion and capable of accommodating the selected active agent(s), which may be micronized, dispersed, suspended or dissolved therein. The vehicle may include pharmaceutically-acceptable emollients, moisturizers, including lactic acid, ammonium lactate and urea, coloring agents, fragrances, emulsifiers, thickening agents, vegetable oils, essential oils, zinc oxide and solvents. Each possibility represents a separate embodiment of this invention.

### Definitions

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

The term “about” as used herein means within an acceptable error range for a particular value as determined by one of ordinary skill in the art, which will depend in part on how the value is measured or determined, i.e., the limitations of the measurement system. For example, “about” can mean a range of up to 10%, more preferably up to 5%, and still more preferably up to 1% of a given value. Where particular values are described in the application and claims, unless otherwise stated, the meaning of the term “about” is within an acceptable error range for the particular value.

The terms “comprise”, “comprising”, “includes”, “including”, “having” and their conjugates mean “including but not limited to”.

The term “consisting of” means “including and limited to”.

The term “consisting essentially of” means that the composition, method or microcapsules may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

In the examples below, all % values referring to a solution are in (w/w). All % values, referring to dispersions (suspensions) are in (w/w). Unless otherwise indicated, all solutions used in the example below refer to an aqueous solution of the indicated ingredient.

### Example 1

### Preparation and Stability of a 0.75% Topical Erlotinib HCl Gel Composition

0.75% erlotinib; 70% DMSO.

### Composition:

| Ingredient | % in formulation |
|---|---|
| Erlotinib hydrochloride | 0.75 |
| DMSO | 70 |
| Propylene glycol | 25.50 |
| 2-phenoxyethanol | 0.5 |
| Methylparaben | 0.25 |
| Carbopol 980 | 3 |

### Procedure:

Erlotinib hydrochloride was dissolved in DMSO at 40° C. Methylparaben was added under stirring. Carbopol was added under stirring. 2-phenoxyethanol was dissolved in propylene glycol and added. The formulation was stirred and homogenized to obtain a homogeneous gel.

### Stability Results:

| | | 1 month | 2 months | 3 months |
|---|---|---|---|---|
| | Time zero | at 40° C. | at 40° C. | at 40° C. |
| Erlotinib assay | 0.70% | 0.71% | 0.71% | 0.73% |

### Example 2

### Preparation and Stability of a 0.5% Topical Erlotinib HCl Gel Composition

0.5% erlotinib; 70% DMSO.

### Composition:

| Ingredient | % in formulation |
|---|---|
| Erlotinib hydrochloride | 0.5 |
| DMSO | 70 |
| Propylene glycol | 25.75 |
| 2-phenoxyethanol | 0.5 |
| Methylparaben | 0.25 |
| Carbopol 980 | 3 |

### Procedure:

Erlotinib hydrochloride was dissolved in DMSO at 40° C. Methylparaben was added under stirring. Carbopol was added under stirring. 2-phenoxyethanol was dissolved in propylene glycol and added. The formulation was stirred and homogenized to obtain a homogeneous gel.

### Stability Results:

| | | 2 months | 3 months |
|---|---|---|---|
| | Time zero | at 40° C. | at 40° C. |
| Erlotinib assay | 0.46% | 0.47% | 0.46% |

### Example 3

### Preparation and Stability of a 0.5% Topical Erlotinib HCl Gel Composition

0.5% erlotinib; 45.5% DMSO

### Composition:

| Ingredient | % in formulation |
|---|---|
| Erlotinib hydrochloride | 0.5 |
| DMSO | 45.5 |
| Propylene glycol | 50.25 |
| 2-phenoxyethanol | 0.5 |
| Methylparaben | 0.25 |
| Carbopol 980 | 3 |

### Procedure:

Erlotinib hydrochloride was dissolved in DMSO at 40° C. Methylparaben was added under stirring. Carbopol was added under stirring. 2-phenoxyethanol was dissolved in propylene glycol and added. The formulation was stirred and homogenized to obtain a homogeneous gel.

### Stability Results:

| | | 2 months | 3 months |
|---|---|---|---|
| | Time zero | at 40° C | at 40° C. |
| Erlotinib assay | 0.47% | 0.47% | 0.46% |

### Example 4

### Preparation and Stability of a 0.5% Topical Erlotinib HCl Gel Composition

0.5% erlotinib; 50% EtOH 70%

### Composition:

| Ingredient | % in formulation |
|---|---|
| Erlotinib hydrochloride | 0.5 |
| EtOH 70% | 50 |
| Propylene glycol | 46.25 |
| 2-phenoxyethanol | 0.5 |
| Methylparaben | 0.25 |
| Carbopol 980 | 2.5 |

### Procedure:

Erlotinib hydrochloride was dissolved in EtOH at 40° C.

Methylparaben was added under stirring. Carbopol was added under stirring. 2-phenoxyethanol was dissolved in propylene glycol and added. The formulation was stirred and homogenized to obtain a homogeneous gel

### Stability Results:

| | | 2 weeks |
|---|---|---|
| | Time zero | at 40° C. |
| Erlotinib assay | 0.47% | 0.48% |

### Example 5

### Preparation and Stability of a 1.25% Topical Erlotinib HCl Gel Composition

1.25% erlotinib; 95% DMSO;

### Composition:

| Ingredient | % in formulation |
|---|---|
| Erlotinib hydrochloride | 1.25 |
| DMSO | 95 |
| 2-phenoxyethanol | 0.5 |
| Methylparaben | 0.25 |
| Carbopol 980 | 3 |

### Example 6

### Preparation and Stability of a 1% Topical Erlotinib HCl Gel Composition

1% erlotinib; 49% PEG-400; 30% PEG-3350

### Composition:

| Ingredient | % in formulation |
|---|---|
| Erlotinib hydrochloride | 1 |
| Propylene glycol | 20 |
| PEG-400 | 49 |
| PEG-3350 | 30 |

### Procedure:

Propylene glycol, PEG-400 and PEG-3350 were stirred at 70% to obtain a homogeneous liquid. Erlotinib hydrochloride was added under stirring. Carbopol was added under stirring and homogenization. 2-phenoxyethanol was dissolved in propylene glycol and added. The formulation was cooled to room temperature.

### Example 7

### Preparation and Stability of a 1% Erlotinib HCl+1% Tapinarof Topical Gel

### Composition:

| Ingredient | % in formulation |
|---|---|
| Erlotinib hydrochloride | 1 |
| Tapinarof | 1 |
| DMSO | 70 |
| Propylene glycol | 24.25 |
| 2-phenoxyethanol | 0.5 |
| Methylparaben | 0.25 |
| Carbopol 980 | 3 |

### Procedure:

Erlotinib hydrochloride is dissolved in DMSO at 40° C. Tapinarof is added under stirring. Methylparaben is added under stirring. Carbopol is added under stirring. 2-phenoxyethanol is dissolved in propylene glycol and added. The formulation is stirred and homogenized to obtain a homogeneous gel.

### Example 8

### Preparation of a 1% Erlotinib HCl+0.5% Tofacitinib Citrate Topical Gel

### Composition:

| Ingredient | % in formulation |
|---|---|
| Erlotinib hydrochloride | 1 |
| tofacitinib citrate | 0.5 |
| DMSO | 70 |
| Propylene glycol | 24.75 |
| 2-phenoxyethanol | 0.5 |
| Methylparaben | 0.25 |
| Carbopol 980 | 3 |

### Procedure:

Erlotinib hydrochloride is dissolved in DMSO at 40° C. Tofacitinib citrate is added under stirring. Methylparaben is added under stirring. Carbopol is added under stirring. 2-phenoxyethanol is dissolved in propylene glycol and added. The formulation is stirred and homogenized to obtain a homogeneous gel.

### Example 9

### Preparation of a 1% Erlotinib HCl+0.5% Apremilast Topical Gel Composition

### Composition:

| Ingredient | % in formulation |
|---|---|
| Erlotinib hydrochloride | 1 |
| Apremilast | 0.5 |
| DMSO | 70 |
| Propylene glycol | 24.75 |
| 2-phenoxyethanol | 0.5 |
| Methylparaben | 0.25 |
| Carbopol 980 | 3 |

### Procedure:

Erlotinib hydrochloride is dissolved in DMSO at 40° C. Apremilast is added under stirring. Methylparaben is added under stirring. Carbopol is added under stirring. 2-phenoxyethanol is dissolved in propylene glycol and added. The formulation is stirred and homogenized to obtain a homogeneous gel.

### Example 10

### PPK Treatment Using Erlotinib Hydrochloride 0.75% Gel

### Study Design

This is a two-part study. The first part is a single blind, within subject vehicle-controlled study and the second part is 12 weeks follow-up phase. In the first part, 18 years of age subjects are admitted into the study only after being genetically confirmed with punctate palmoplantar keratoderma mutation (AAGAB mutation). Eligible subjects are enrolled for a daily treatment with Erlotinib Hydrochloride Gel 0.75%, and its vehicle gel. In the second part, subjects are subjected for evaluation every 4 weeks for additional 12 weeks.

### Dosage

Subjects apply the study product once daily for 12 weeks along the entire plantar. Subjects apply the study product on designated leg and the vehicle product on the other leg.

### Clinical Outcome Assessments

The determination of efficacy is based on improvement in the disease's signs and symptoms severity. The symptoms are subjected for evaluation by both principal investigator and the subject. The principal investigator determines signs/symptoms such as thickness measurement of the outer layer skin of the feet soles through ultrasound. Treatment efficacy measured by subject includes subject report on improvement according to the pain and PRO (Patient Reported Outcome; see below) questionnaires over time vs the vehicle control. Additional assessment on the feet is done by the principal investigator by scoring the degrees of hyperkeratosis or callosities, fissure, thickening, roughness and scaling on a four-point scale [0 (None); −1 (Mild); 2 (Moderate) and 3 (Severe)] at all study visits beginning at Baseline (after the debridement procedure).

### Investigator Cutaneous Safety Assessment

Investigator Cutaneous Safety Assessment is performed at each study visit starting baseline, and assessing the local treatment area cutaneous reactions, by rating the: dryness and scaling, on a scale ranging from 0 (Absent) to 3 (Severe). The evaluator determines the score for each of the variables by direct evaluation:

### Investigator Assessment of Side Effects

Investigator assess the presence or absence of EGFR inhibitor common side effects of Xerosis, Papulopustular skin eruptions and Paronychia at each study visit.

### Subject Assessment of Local Tolerability

At each study visit starting baseline, the subject is asked to assess the Local Tolerability of the treatment area, by rating of, itching and burning/stinging, on a scale ranging from 0 (Absent) to 3 (Severe). The subject is asked to grade each of the variables based on their experience over the past 24 hours.

What is claimed is:

1. A method of treatment, prevention or alleviation of Palmoplantar Keratoderma (PPK) or Olmsted syndrome in a patient in need thereof, comprising:
a. debridement of affected PPK surface area or Olmsted syndrome surface area of said patient; and
b. topical administration, following the debridement of step (a), of a therapeutically effective amount of a topical composition comprising at least one Epidermal Growth Factor Receptor (EGFR) inhibitor, to said affected surface areas.

2. The method of claim 1, wherein said topical composition is administered for at least one week.

3. The method of claim 2, wherein said topical composition is administered once daily for 12 weeks.

4. The method of claim 1, wherein said debridement is performed by physical, chemical/pharmacological or mechanical removal of nonviable or unwanted tissue affected by the PPK or by the Olmsted syndrome.

5. The method of claim 4, wherein said physical or mechanical removal comprises using scalpel, monofilament pads, forceps or scissors.

6. The method of claim 4, wherein the chemical/pharmacological removal of nonviable or unwanted tissue affected by the PPK or by the Olmsted syndrome comprises oral treatment of at least one EGFR inhibitor or by applying a chemical agent to the PPK surface area or the Olmsted syndrome surface area, or by combination thereof.

7. The method of claim 1, wherein said debridement comprises skin graft techniques.

8. The method of claim 7, wherein said skin graft techniques are combined with or performed following application of a chemical agent to said affected PPK surface area or the Olmsted syndrome surface area.

9. The method of claim 4, wherein said physical, chemical/pharmacological or mechanical removal is performed following application of a chemical agent to said affected PPK surface area or the Olmsted syndrome surface area.

10. The method of claim 1, wherein said debridement step comprises ultrasonication combined with applying of a chemical agent to said affected PPK surface area or the Olmsted syndrome surface area.

11. The method of claim 1, wherein said debridement comprises applying a chemical agent.

12. The method of claim 9, wherein said chemical agent comprises urea, salicylic acid, lactic acid, retinoid, psoralen, corticosteroids, debriding agent, derivatives thereof or any combination thereof.

13. The method of claim 11, wherein said chemical agent comprises urea, salicylic acid, lactic acid, retinoid, psoralen, corticosteroids, debriding agent, derivatives thereof or any combination thereof.

14. The method of claim 12, wherein said chemical agent comprises a combination of urea, salicylic acid, lactic acid, retinoid, psoralen, corticosteroids or any combination thereof with a debriding agent.

15. The method of claim 13, wherein said debriding agent is selected from the group consisting of: debridase, vegetable preparation, bromelain, ananain, cysteine protease precursor and any combination thereof.

16. The method of claim 14, wherein said debriding agent is selected from the group consisting of: debridase, vegetable preparation, bromelain, ananain, cysteine protease precursor and any combination thereof.

17. The method according to claim 9, wherein said topical composition is a combination product comprising said EGFR and said chemical agent; or wherein said EGFR and said chemical agent are administered sequentially or concomitantly.

18. The method of claim 1, wherein said topical composition further comprises a therapeutically effective amount of at least one penetration enhancer.

19. The method of claim 18, wherein the at least one penetration enhancer is selected from the group consisting of: DMSO, ethanol, isopropyl alcohol, dimethyl isosorbide, isopropyl myristate, oleic acid, a polyethylene glycol, hexylene glycol, glycofurol and combinations thereof.

20. The method of claim 19, wherein the amount of the at least one penetration enhancer is from about 10% to about 98% w/w of at least one penetration enhancer.

21. The method of claim 1, wherein the at least one EGFR inhibitor is selected from the group consisting of: erlotinib, gefitinib, lapatinib, osimertinib, brigatinib their salts, hydrates or solvates and combinations thereof.

22. The method of claim 21, wherein the at least one EGFR inhibitor is erlotinib hydrochloride.

23. The method according to claim 1, wherein the amount of the at least one EGFR inhibitor is from about 0.1% w/w to about 1% w/w, from about 1% w/w to about 3% w/w, from about 3% w/w to about 5% w/w or from about 5% w/w to about 10% w/w.

24. The method of claim 23, wherein the at least one EGFR inhibitor is erlotinib hydrochloride and the amount thereof is 0.75% w/w.

25. The method of claim 1, wherein said topical composition further comprises at least one ingredient selected from the group consisting of: a moisturizer, a skin barrier, urea, ammonium lactate and combinations thereof, in a concentration of from about 0.01% w/w to about 1% w/w, from about 1% w/w to about 3% w/w or from about 3% w/w to about 5% w/w.

26. The method of claim 1, wherein said topical composition further comprises at least one additional active agent selected from the group consisting of: tapinarof, a Janus kinase inhibitor (JAK inhibitor), a phosphodiesterase-4 inhibitor (PDE4 inhibitor), a corticosteroid, calcipotriene and combinations thereof, in a concentration of from about 0.01% w/w to about 1% w/w, from about 1% w/w to about 3% w/w or from about 3% w/w to about 5% w/w.

27. The method of claim 1, wherein said topical composition further comprises at least one additional active agent selected from the group consisting of: menadione, ketoconazole, dapsone, cevimeline, spironolactone, retinoid, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% w/w to about 1% w/w, from about 1% w/w to about 3% w/w, from about 3% w/w to about 5% w/w or from about 5% w/w to about 10% w/w.

28. The method of claim 1, wherein said composition is a gel, a hydrogel, a cream, an ointment, a lotion, a spray, a shampoo, a patch or a foam.

29. The method of claim 1, wherein the at least one EGFR inhibitor is partly or entirely solubilized in said composition.

30. The method of claim 1, wherein the at least one EGFR inhibitor is erlotinib hydrochloride and the composition is formulated as a topical gel.

31. The method of claim 1, wherein the topical composition comprises about 0.75% w/w erlotinib hydrochloride and from about 10% to about 98% w/w of at least one penetration enhancer, and wherein the composition is formulated as a gel.

32. The method of claim 31, comprising about 0.75% w/w erlotinib hydrochloride, about 70% w/w DMSO, about 25% propylene glycol, about 0.5% w/w 2-phenoxyethanol, about 0.25% w/w methylparaben and about 3% w/w Carbopol 980, wherein the composition is formulated as a gel.

33. The method of claim 26, wherein said at least one EGFR inhibitor and said at least one additional active agent exhibit an additive or synergistic effect.

34. The method of claim 1, wherein the treatment, prevention or alleviation of PPK or Olmsted syndrome by topical administration of a composition comprising at least one EGFR inhibitor does not induce or induces reduced cutaneous side-effects as compared with systemic administration of the same EGFR inhibitor amount.

35. The method of claim 1, wherein said PPK is acquired or hereditary.

36. The method of claim 1, wherein said PPK is diffuse, focal, striate or punctate PPK.

37. The method of claim 36, wherein said punctate PPK is a Punctate palmoplantar keratoderma type 1 (PPPK-1).
